(19) United States
(12) Patent Application Publication
(10) Pub. No.: US 2025/0011850 A1
(43) Pub. Date: Jan. 9, 2025

(54) POLYMERASE MUTANT AND APPLICATIONS THEREOF

(71) Applicants: Shanghai Zhongqi Biotechnology Co., Ltd., Shanghai (CN); Shanghai Xiansai Biotechnology Co., Ltd., Shanghai (CN)

(72) Inventors: Xuejun HU, Shanghai (CN); Liqin CAO, Shanghai (CN)

(21) Appl. No.: 18/885,661

(22) Filed: Sep. 15, 2024

Related U.S. Application Data
(63) Continuation of application No. PCT/CN2023/095944, filed on May 24, 2023.

(30) Foreign Application Priority Data
May 9, 2022 (CN) 202210495569.4

(51) Int. Cl. C12Q 1/6844; C12N 9/12

(52) U.S. Cl. CPC C12Q 1/6844; C12N 9/1252; C12Y 207/07007

(57) ABSTRACT

A ZO5 DNA polymerase mutant and its application are disclosed. The ZO5 DNA polymerase mutant is obtained by mutation of a ZO5 wild-type polymerase, which has the amino acid sequence shown as SEQ ID NO:1. Mutation sites include E628K, I709L, E744R, and A745R. The polymerase mutant can expand its capacity for a variety of activities, including reverse transcriptase, and can catalyze reverse transcription-loop mediated isothermal amplification using an RNA template. The polymerase mutant after fusion of the binding peptide has a strong ability to resist interference, such as chocolate, peanut butter, milk, seafood, meat or egg, chocolate, pepper, blood, urine, humic acid, bile, tannin, melanin, indigo dye, plant materials, etc., and can effectively shorten the LAMP required time.

### CROSS-REFERENCE TO RELATED APPLICATIONS

This application is a continuation of International Pat. Appl. No. PCT/CN2023/095944, filed on May 24, 2023, which claims priority to Chinese Pat. Appl. No. 202210495569.4, filed on May 9, 2022, the contents of each of which are incorporated by reference herein in their entireties.

### REFERENCE TO AN ELECTRONIC SEQUENCE LISTING

The contents of the electronic sequence listing (SHCH-2200291-US.xml; Size: 29,078 bytes; and Date of Creation: Sep. 15, 2024) is incorporated herein by reference in its entirety.

### TECHNICAL FIELD

The invention belongs to the field of molecular biology, in particular to a polymerase mutant and applications thereof.

### BACKGROUND

Taq polymerase and Bst polymerase are two well-known heat-resistant DNA polymerases with 50% sequence homology in their crystal structures. Taq polymerase can catalyze the polymerase chain reaction (PCR) (this reaction requires that the enzyme must tolerate 94° C.), but Bst polymerase activity is at 65˜70° C., so it cannot catalyze PCR. At the same time, Taq polymerase cannot replicate double-stranded DNA by replacing the previous DNA strand, and the 5′-endonuclease of Taq polymerase can degrade the replaced DNA. However, Bst polymerase can efficiently perform chain displacement without degradation, and is therefore commonly used to catalyze loop-mediated isothermal amplification (LAMP). But neither Taq polymerase nor Bst polymerase has reverse transcriptase (RT) activity, so it is usually necessary to combine a separate RT enzyme with them to complete RT-PCR (reverse transcriptase polymerase chain reaction) or a similar RT-LAMP.

In contrast, ZO5 DNA polymerase has strong reverse transcriptase polymerase chain reaction (RT-PCR) activity, but cannot replicate double-stranded DNA by chain replacement, and therefore cannot be used to catalyze loop-mediated isothermal amplification (LAMP).

In order to amplify an RNA target by PCR or LAMP, it is necessary to first reverse transcribe the RNA template into cDNA. In RT-PCR or RT-LAMP, it is difficult for mixed enzymes to exert their respective advantages, due to their different optimal reaction conditions. Typically, RT-PCR assays rely on non-heat-stable reverse transcriptase (RNA-dependent DNA polymerase) derived from thermophilic organisms for the initial cDNA synthesis step (RT). In PCR, an additional heat-stable DNA polymerase is required for cDNA amplification to tolerate the temperature increase required for nucleic acid denaturation.

### SUMMARY

The present invention aims to provide a DNA polymerase mutant and its application.

To fulfill the purpose of the invention, the technical scheme adopted by the invention includes a ZO5 DNA polymerase mutant obtained by mutation of a ZO5 wild-type polymerase having the amino acid sequence shown in SEQ ID NO: 1, including the mutations E628K, I709L, E744R and A745R.

Preferably, the mutation sites also include E710K, E710L, E710N, E710Q, E710I, E710W, E710R, E710V or E710S.

Preferably, the polymerase mutant is obtained by truncating the wild-type polymerase.

Preferably, truncation is performed at site 282 of the wild-type polymerase.

Preferably, the polymerase mutant further includes a binding peptide bound or fused thereto.

Preferably, the binding peptide is selected from Sso 7d, Tm Csp, and Sac 7d.

Correspondingly, the invention further includes a polymerase mutant whose amino acid sequence is shown as SEQ ID NO: 3.

Correspondingly, the invention further includes a polymerase mutant whose amino acid sequence is shown as SEQ ID NO: 7.

Accordingly, the polymerase mutant is applied to PCR, RT-PCR, LAMP and RT-LMAP.

Correspondingly, the invention further includes reagents, test strips and kits containing the polymerase mutant(s).

The invention has the following beneficial effects. The use of engineered thermally active or thermally stable DNA polymerases for more efficient reverse transcription for RT-PCR or similar RT-LAMP assays has several potential benefits.

1. Enhanced reverse transcriptase activity together with the ability to use higher reverse transcriptase incubation temperatures, which allow relaxation of the RNA template secondary structure, and can lead to overall higher cDNA synthesis efficiency and detection sensitivity.

2. Higher temperature incubation may also increase specificity by reducing mismatches in the reverse transcription step.

3. Enzymes with improved reverse transcriptional efficiency can simplify assay design by reducing RT incubation time and/or enzyme concentration(s).

4. When dUTP and UNG are used (e.g., in or for PCR), non-specific extension products containing dUMP formed during PCR under non-rigorous conditions are degraded by UNG, and cannot be used as primers or templates. It is not possible to utilize the dUTP and UNG methods when using non-heat-stable reverse transcriptase (RNA-dependent DNA polymerase) derived from mesophilic organisms. However, the use of the present thermally-active or thermally-stable DNA polymerase for the reverse transcription step makes the reaction fully compatible with the use of a dUTP/uracil n-glycosylase (UNG) residue prevention system. In addition to providing a residue contamination control, the use of dUTP and UNG provides a “hot start” to reduce nonspecific amplification.

The polymerase mutant provided by the invention can expand its ability to perform a variety of activities, including reverse transcriptase (RT), and can catalyze reverse transcription-loop mediated isothermal amplification (RT-LAMP) using an RNA template. The polymerase mutant, after fusion of the binding peptide, also has a strong ability to resist adverse interference from contaminants, such as chocolate, peanut butter, milk, seafood, meat or egg, chocolate, pepper, blood, urine, humic acid, bile, tannin, melanin, indigo dye, plant materials, etc., and can effectively shorten the time for the LAMP process.

Contamination problems in RT-LAMP can also be solved by using the polymerase mutant provided by the invention. The biggest disadvantage of loop-mediated isothermal amplification method is that due to its very high sensitivity, once the lid is opened, it is easy to form aerosol pollution, and false positives become a serious problem. The problem can be solved when primers are prepared and diluted in an uncontaminated environment and added to the lid of each reaction tube. The remaining components of the reaction (buffer, DNA polymerase, MS2 template, etc.) can be added to the reaction tube with RNase-free DNase I and MgCl₂. The reaction can be conducted at room temperature for a period of time sufficient to degrade the DNA sample under the action of DNase I, and then the reaction tube can be gently moved to the thermal cycler with the primer still hanging on the lid. After heating on the heating block for some time, DNase I loses its activity. The reaction tube can then be removed from the heating block and mixed, then immediately returned to the heating block for incubation at a set temperature. The recognized pollution problem of the RT-LAMP reaction can be completely or partially overcome by using the above method.

### BRIEF DESCRIPTION OF THE DRAWINGS

FIG. 1 shows the spatial structure diagram of wild-type polymerase and corresponding mutation sites.

FIG. 2 shows the results of an RT-PCR experiment using one of the present polymerase mutants.

FIG. 3 shows the results of an RT-LAMP experiment using one of the present polymerase mutants.

FIG. 4 shows results of a LAMP anti-interference experiment using the polymerase mutant ZO5 L2.

### DETAILED DESCRIPTION OF THE EMBODIMENTS

A DNA polymerase (ZO5 polymerase) mutant is provided. The DNA polymerase mutant is a mutated ZO5 wild-type polymerase. The amino acid sequence of the wild-type polymerase is given in SEQ ID NO: 1, and a corresponding DNA sequence (e.g., of the wild-type ZO5 polymerase) is given in SEQ ID NO: 2. The polymerase mutant was obtained by mutating E628K, I709L, E710K, E744R and A745R in the ZO5 wild-type polymerase. An alternative scheme preserves the E628K, 1709L, E744R and A745R mutations, and further includes the mutation E710L, E710N, E710Q, E710I, E710W, E710R, E710V, or E710S for site 710.

The optimal scheme is as follows. The polymerase mutant is obtained by truncating the wild-type polymerase, and the truncation site can occur at any site of the full-length sequence, as long as the activity of ZO5 polymerase is preserved. It is preferred to truncate at site 282 of the wild-type polymerase of ZO5; that is, the first site of the truncated polymerase mutant corresponds to Site 282 of the wild-type polymerase amino acid sequence (SEQ ID NO: 1).

Another preferred scheme is that the polymerase mutant can further include one or more of Sso 7d, Tm Csp, Sac 7d and other binding peptides. Fusion binding peptides such as Sso7d and Sso7d binding to DNA will introduce a negative superhelix to DNA. Increasing the melting temperature of DNA can enhance the anti-interference ability of the present polymerase mutants and effectively shorten the LAMP time.

It should be understood that persons skilled in the art may, as needed, first perform mutations at the corresponding sites and then truncate the ZO5 polymerase, or first truncate the ZO5 polymerase and then perform mutations at the corresponding sites. Polymerase mutants can be obtained by directly synthesizing the corresponding amino acid sequence, or by synthesizing the corresponding DNA sequence and then translating it into amino acid sequence, or by truncating and/or mutating the wild-type polymerase.

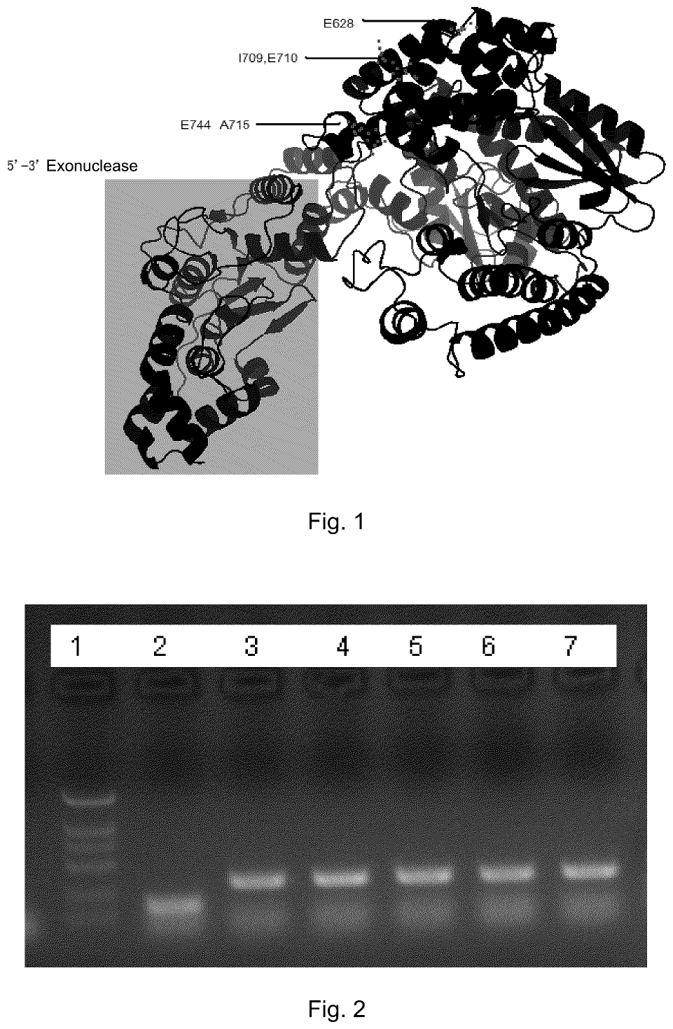

After the wild-type polymerase is truncated at the Site 282 and mutated to include the E628K, I709L, E710K, E744R and A745R mutations, the amino acid sequence shown in SEQ ID NO: 3 was obtained for the polymerase mutant. The spatial structure diagram of wild-type polymerase and corresponding mutation sites is shown in FIG. 1.

The following is a clear and complete description of the technical scheme in embodiments of the invention. Obviously, the embodiments described are only part of the embodiments of the invention, but not all of the possible embodiments. Unless otherwise specified, the technical means used in the embodiments are conventional and well-known to those skilled in the art. All of the data presented herein are the average values obtained after at least 3 repetitions, and all of the data obtained from each repetition used in the average value calculation are valid data.

### EXAMPLE 1: EXPRESSION AND PURIFICATION OF POLYMERASE MUTANTS

Step (1): Preparation of a polymerase mutant gene expression vector. The plasmid template of the ZO5 wild-type polymerase gene (e.g., including SEQ ID NO: 2) was selected, and E628K-F, E628K-R, IE709LK-F, IE709LK-R, EA744RR-F, and EA744RR-R were used as mutation primers. ZO5-Nde1-F and ZO5-Sal1-R were used as truncated PCR primers. 1 μL of the primers (10 μM) were added to a 50 μL reaction system and mixed with a 50 μL PCR reaction system, including 10×buffer (100 mM KCl, 100 mM (NH₄)₂SO₄, 200 mM Tris-HCl [pH=8.8], 20 mM MgSO₄, 5 μL 1% TritonX-100, 1 mg/mL BSA), 3 μL 2 mM dNTP, and 1 μL of the KOD enzyme. A total of 25 amplification cycles were performed according to the following procedure: 95° C., 1 min; 95° C., 30 s; 60° C., 30 s; 68° C., 2 min; 68° C., 5 min; and then storing at 4° C. The amplification primers are shown in Table 1. IE709LK stands for I709L, and E710K and EA744RR stand for E744R and A745R.

TABLE 1
Sequence of amplified primers

| Primer | Sequence |
|---|---|
| E628K-F | CTCATCTGAGCGGTGACAAGAACCTGATTCGTGTGTTTCA |
| | (SEQ ID NO: 9) |
| E628K-R | CTTTGTGTGCTTAGTCCAAGAACAGTGGCGAGTCTACTCG |
| | GTCTTG (SEQ ID NO: 10) |
| IE709LK- | GAAAGTTCGTGCGTGGCTCAAAAAAACCCTGGAAGAAGGT |
| F | CG (SEQ ID NO: 11) |
| IE709LK- | GAAGAAGGTCCCAAAAAAACTCGGTGCGTGCTTGAAAGCC |
| R | CTTCCTG (SEQ ID NO: 12) |
| EA744RR- | GTTAAATCTGTACGCCGCCGTGCTGAACGTATGGCATTTA |
| F | ACATG (SEQ ID NO: 13) |
| EA744RR- | TACGGTATGCAAGTCGTGCCGCCGCATGTCTAAATTGTGC |
| R | GCGCAAG (SEQ ID NO: 14) |
| ZO5- | TAAGAAGGAGATATACATATGGGTCTGCTGCACGAGTTTG |
| Nde1-F | GCCTGC (SEQ ID NO: 15) |
| ZO5- | GCTACCACCGCCCCCGTCGACACCTTTAGCGCTCAGCCAA |
| Sal1-R | TCTTCAC (SEQ ID NO: 16) |

The amplified reaction system was subject to electrophoresis with 1.2% agarose gel at 150 V for 30 min, and the target strip was recovered. The pET 42a vector treated with Nde1 and Xho1 was connected by an enzyme-ligating method, and the clone was selected for sequencing. The DNA sequence obtained was SEQ ID NO: 4. Seamless cloning was adopted to connect the pET Sso 7d vector treated with Nde1 and Sal1 (from Shanghai Xiansai Biotechnology Co., Ltd., containing binding peptide Sso 7d), then the clone was selected and sequenced. The obtained DNA sequence is SEQ ID NO: 6.

Step (2): Expression and purification of the polymerase mutants. The two plasmid vectors obtained in step (1) were respectively introduced into BL21 (DE3) and cultured overnight in an LB medium containing antibiotics at 37° C., 160 r/min. Escherichia coli bodies obtained after cloning-induced expression were selected respectively. Two polymerase mutants were obtained after purification. The amino acid sequence obtained using SEQ ID NO: 4 was SEQ ID NO: 5, and named ZO5 L1. SEQ ID NO: 6 corresponds to the amino acid sequence SEQ ID NO: 7, named ZO5 L2. To be clear: In the amino acid sequences of ZO5 L1 and ZO5 L2 (SEQ ID NO: 5, SEQ ID NO: 7), “Leu Glu His His His His His His His (His)” at the end of the sequences came from the carrier, which had no effect on the performance of the polymerase mutant. These end sequences can be kept or omitted. ZO5 L2 is based on ZO5 L1, but with the binding peptide Sso 7d.

### EXAMPLE 2: RT-PCR USING POLYMERASE MUTANTS

Using the DNA sequence of MS2 in the Chinese patent publication no. 113846146A as a template (the DNA sequence is shown in SEQ ID NO: 8), each hole contains 3 μL ZO5 L1 and ZO5 L2 enzymes diluted 10 times with buffer solution, and ZO5 wild-type polymerase is used as a control. PCR and RT-PCR were performed. In each group, the buffer contains 20 mM Tris-HCl (pH=8), 100 mM KCl, 0.1 mM EDTA, and 0.1% Tween-2. They were added to 12 μL RT-PCR premixes as shown in Table 2 for thermal cycling. Heat cycle conditions included 50° C., 2 minutes (the “UNG” step); 65° C., 2 minutes (the “RT” step); 5 cycles at 94° C., 15 seconds; then 62° C., 30 seconds; then 45 cycles at 91° C. for 15 seconds; then 62° C. for 30 seconds.

TABLE 2
RT-PCR premixes

| Constituent | Concentration | |
|---|---|---|
| Tris-HCl (pH = 8) | 50 | mM |
| KOAc | 60 | mM |
| glycerin | 5% | (v/v) |
| DMSO | 2% | (v/v) |
| MgCl₂ | 2 | mM |
| primer 1 | 200 | nM |
| primer 2 | 200 | nM |
| dUTP | 400 | nM |
| dATP | 200 | nM |
| dCTP | 200 | nM |
| dGTP | 200 | nM |
| UNG | 2 | U/μL |
| MS2 RNA Template | 50 | ng |

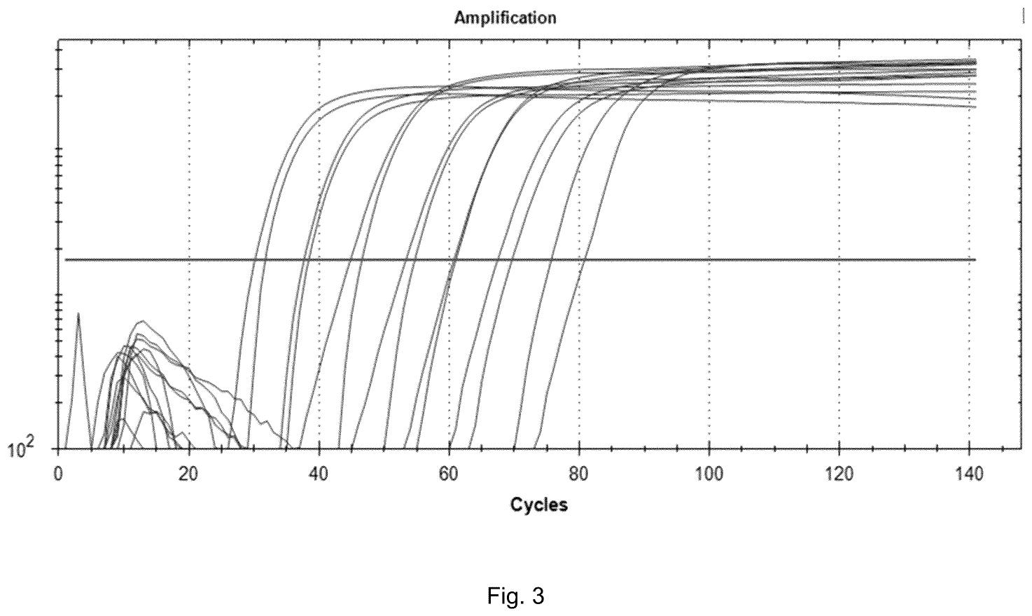

The result is shown in FIG. 2. In FIG. 2, lane 1 represents DL2000 DNA markers, lane 2 is the blank/control, lanes 3 and 4 are ZO5 L1, lanes 5 and 6 are ZO5 L2, and lane 7 is ZO5 wild-type polymerase. The results showed that ZO5 L1 and ZO5 L2 retained the basic properties of ZO5 wild-type polymerase for RT-PCR.

### EXAMPLE 3: LAMP REACTION TEMPERATURE TEST OF POLYMERASE MUTANTS

Primers were designed to detect MS2 specific genes (the MS2 gene sequence is shown in SEQ ID NO: 8), the specific sequence of designed MS2 primers is shown in Table 3,and the reaction system is shown in Table 4.

TABLE 3
Sequence of MS2 primers

| Primers | Sequences (5′ to 3′) |
|---|---|
| MS2-BIP | GCACGTTCTCCAACGGTGCTGGTTGCTTGTTCAGCGAACT |
| | (SEQ ID NO: 17) |
| MS2-FIP | GCCCAAACAACGACGATCGGTAAAACCAGCATCCGTAGCCT |
| | (SEQ ID NO: 18) |
| MS2-LB | TGCAGGATGCAGCGCCTTA (SEQ ID NO: 19) |
| MS2-LF | CCAGAGAGGAGGTTGCCAA (SEQ ID NO: 20) |
| MS2-B3 | CAATAGAGCCGCTCTCAGAG (SEQ ID NO: 21) |
| MS2-F3 | TGTCATGGGATCCGGATGTT (SEQ ID NO: 22) |

TABLE 4
Reaction system

| Samples | Concentration | | Amount added (μL) |
|---|---|---|---|
| MS2 template | 1 | ng/uL | 1 |
| MS2-BIP | 10 | μm | 1.6 |
| MS2-FIP | 10 | um | 1.6 |
| MS2-LF | 10 | um | 0.8 |
| MS2-LB | 10 | um | 0.8 |
| MS2-F3 | 10 | um | 0.2 |
| MS2-B3 | 10 | um | 0.2 |
| dNTP/dUTP Mixture | 25 | mM | 0.8 |
| Mg2+ | 100 | mM | 1 |

| | | |
|---|---|---|
| 10× buffer | \ | 2.5 |
| DNA polymerase | \ | 1 |
| Eva Green Dye | 10× | 2.5 |
| Water | \ | 10.5 |
| UDG | | 1 |
| Bulk volume | \ | 25 uL |

The above reaction system was carried out in a Bio-Rad CFX Connect™ PCR system using a SYBR program, and the Plate Read was performed at 60° C. and 30 s for 120 cycles. The Gradient Range was set to 15.0. LAMP expansion was performed at A: 75.0° C., B: 74.2° C., C: 72.5° C., D: 69.5° C., E: 65.9° C., F: 63.0° C., G: 61.0° C. and H: 60.0° C. The results are shown in Table 5.

TABLE 5
Comparison table of temperature experiment results

| Temperature | ZO5 L2 Cp | ZO5 L1 Cp |
|---|---|---|
| 75.0° C. | 50.2 | 55.2 |
| 74.2° C. | 37.6 | 40.6 |
| 72.5° C. | 31.6 | 35.7 |
| 69.5° C. | 33.2 | 39.3 |
| 65.9° C. | 55.2 | 60.4 |
| 63.0° C. | 70.2 | N/A |
| 61.0° C. | N/A | N/A |
| 60.0° C. | N/A | N/A |

The results showed that the enzyme activity of the polymerase mutant was the best at 72° C., and the reaction rate of ZO5 L2 was faster than that of ZO5 L1. The results further showed that the polymerase mutant can use a conventional dNTP/dUTP mixture to conduct the experiment, which can reduce or prevent contamination.

### EXAMPLE 4: RT-LAMP EXPERIMENT OF POLYMERASE MUTANTS

Using the conditions of Example 3, a gradient 10-fold dilution of the RNA template for MS2 was prepared in a Bio-Rad CFX Connect™ PCR system using a SYBR program. LAMP extension was then performed at 50° C. for 2 minutes (the “UNG” step); 65° C. for 2 minutes (the “RT” step); 72° C., 30 seconds for 1 cycle, and Plate Read (72° C., 30 seconds), 120 cycles. The results are shown in Table 6 and FIG. 3.

TABLE 6
LAMP results of ZO5 L2 at different
template RNA concentrations

| Template RNA | | |
|---|---|---|
| concentrations | | ZO5 L2 Cp |
| 8 | ng | 30.13 |
| 800 | pg | 38.38 |
| 80 | pg | 46.56 |
| 8 | pg | 54.70 |
| 800 | fg | 61.03 |
| 80 | fg | 67.44 |
| 8 | fg | 80.79 |

| | |
|---|---|
| 0 | N/A |

The results show that ZO5 L2 has better reaction speed, and the RT-LAMP experiment on ZO5 L2 shows very good RT-LAMP performance as shown in Table 6 and FIG. 3.

### EXAMPLE 5: LAMP IMMUNITY EXPERIMENT OF POLYMERASE MUTANT ZO5 L2

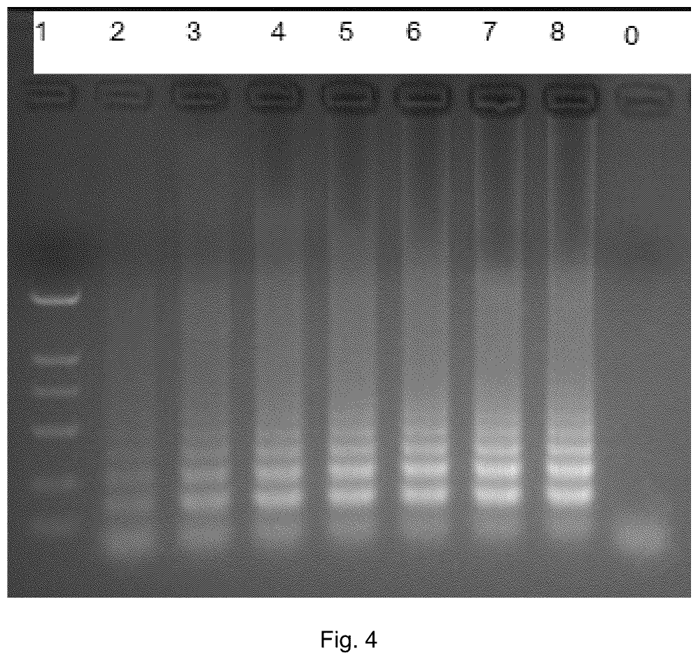

Under the conditions of Example 4, the DNA template of MS2 (200 pg) was added into a reaction tube, and the reaction was carried out in a Bio-Rad CFX Connect™ PCR system using a SYBR program, with a cycle of 72° C., 30 seconds and Plate Read, 120 cycles, setting multiple processes. LAMP expansion was performed by adding different interfering substances (10% urine, 10% whole blood, 10% E. coli breakdown solution). The results are shown in FIG. 4.

In FIG. 4, lane 1 is DL2000 DNA markers, lane 0 is a blank/control, lanes 2 and 3 are treatments with 10% urine added, lanes 4 and 5 are treatments with 10% whole blood added, lanes 6 and 7 are treatments with 10% E. coli breakdown solution added, and lane 8 is a control treatment with an amount of water equal to the volume of interfering substance added. The results showed that the polymerase mutant ZO5 L2 had good immunity ability.

Example 6: Mutation of the amino acid at position 710 of the polymerase mutant ZO5

Using the ZO5 L2 polymerase with the mutation at site 710 (e.g., E710K, E710L, E710N, E710Q, E710I, E710W, E710R, E710V or E710S), the experiment of Example 4 with a template RNA concentration or mass of 80 pg was repeated, with other conditions unchanged. In addition, 10% whole blood was used as the interfering substance, and the results are shown in Table 7.

TABLE 7
Comparison table of performance display of polymerase mutant
ZO5 after amino acid mutation at Site 710

| Site 710 | | LAMP immunity |
|---|---|---|
| mutation | RT-LAMP cp | experiments active cp |
| E710K | 67.66 | 68.45 |
| E710L | 67.85 | 67.52 |
| E710N | 69.2 | 69.82 |
| E710Q | 68.51 | 69.52 |
| E710I | 67.39 | 68.27 |
| E710W | 70.58 | 66.31 |
| E710R | 69.25 | 67.34 |
| E710V | 68.01 | 71.06 |
| E710S | 67.23 | 69.54 |

The results showed that the mutations E710L, E710N, E710Q, E710I, E710W, E710R, E710V and E710S at Site 710 had the same RT-LAMP ability and anti-interference ability as E710K.

The above embodiments describes only various methods and examples of the invention and do not limit the scope of the invention. Under the premise of not deviating from the design or spirit of the invention, all kinds of deformations, variations, modifications and replacements of the technical scheme of the invention made by ordinary technicians in the field shall fall within the scope of protection determined by the claims of the invention.

What is claimed is:

1. A ZO5 DNA polymerase mutant, obtained by mutation of a ZO5 wild-type polymerase having an amino acid sequence shown in SEQ ID NO:1, including mutations E628K, I709L, E744R and A745R.

2. The polymerase mutant according to claim 1, wherein the mutations also include E710K, E710L, E710N, E710Q, E710I, E710W, E710R, E710V or E710S.

3. The polymerase mutant according to claim 1, wherein the polymerase mutant is obtained by truncating the wild-type polymerase.

4. The polymerase mutant according to claim 3, wherein the polymerase mutant is truncated at site 282 of the wild-type polymerase.

5. The polymerase mutant according to claim 1, further comprising a binding peptide bound to the polymerase mutant.

6. The polymerase mutant according to claim 5, wherein the binding peptide is selected from Sso 7d, Tm Csp and Sac 7d.

7. A polymerase mutant, having an amino acid sequence shown in SEQ ID NO:3.

8. A polymerase mutant, having an amino acid sequence shown in SEQ ID NO:7.

9. Application of the polymerase mutant in claim 1 in PCR, RT-PCR, LAMP, or RT-LMAP.

10. Reagents, test strips and kits containing the polymerase mutant in claim 1.


SEQUENCE LISTING (22 sequences)

SEQ ID NO: 1 - protein, 834 aa, Thermus sp.
MKAMLPLFEPKGRVLLVDGHHLAYRTFFALKGLTTSRGEPVQAVYGFAKSLLKALKEDGYKAVFVVFDAKAPSFRHEAYEAYKAGRAPTPEDFPRQLALIKELVDLLGFTRLEVPGFEADDVLATLAKKAEREGYEVRILTADRDLYQLVSDRVAVLHPEGHLITPEWLWEKYGLKPEQWVDFRALVGDPSDNLPGVKGIGEKTALKLLKEWGSLENILKNLDRVKPESVRERIKAHLEDLKLSLELSRVRSDLPLEVDFARRREPDREGLRAFLERLEFGSLLHEFGLLEAPAPLEEAPWPPPEGAFVGFVLSRPEPMWAELKALAACKEGRVHRAKDPLAGLKDLKEVRGLLAKDLAVLALREGLDLAPSDDPMLLAYLLDPSNTTPEGVARRYGGEWTEDAAHRALLAERLQQNLLERLKGEEKLLWLYQEVEKPLSRVLAHMEATGVRLDVAYLKALSLELAEEIRRLEEEVFRLAGHPFNLNSRDQLERVLFDELRLPALGKTQKTGKRSTSAAVLEALREAHPIVEKILQHRELTKLKNYYVDPLPGLVHPRTGRLHTRFNQTATATGRLSSSDPNLQNIPIRTPLGQRIRRAFVAEAGWALVALDYSQIELRVLAHLSGDENLIRVFQEGKDIHTQTASWMFGVSPEAVDPLMRRAAKTVNFGVLYGMSAHRLSQELAIPYEEAVAFIERYFQSFPKVRAWIEKTLEEGRKRGYVETLFGRRRYVPDLNARVKSVREAAERMAFNMPVQGTAADLMKLAMVKLFPHLREMGARMLLQVHDELLLEAPQARAEEVAALAKEAMEKAYPLAVPLEVEVGIGEDWLSAKG

SEQ ID NO: 2 - DNA, 2502 bp, Thermus sp.
  source 1..2502: mol_type = genomic DNA
atgaaagcgatgctgccgctgttcgaaccgaaaggtcgtgttctgctggttgatggccaccacctggcgtaccgtaccttcttcgcgctgaaaggtctgaccacttctcgtggtgaaccggtacaggcggtctacggcttcgctaaatccctgctgaaagcactgaaagaagatggctataaagctgtatttgtagtatttgacgccaaagcgccgtccttccgtcatgaagcttacgaagcgtacaaagcaggccgtgcgccgaccccggaagatttcccgcgccagctggcgctgattaaagaactggttgatctgctgggtttcactcgtcttgaagttccgggtttcgaagcggatgatgttctggctaccctggcgaaaaaagcggaacgtgaaggctacgaagtgcgtatcctgaccgctgatcgtgacctgtaccagctggtgtctgatcgcgttgctgtgctgcatccggaaggccacttgatcaccccggaatggctgtgggaaaaatacggtctgaaaccggaacagtgggtggatttccgcgcgctggttggcgacccgagcgacaatctgccgggcgtgaaaggtatcggcgaaaaaaccgcactgaaactgctgaaagaatggggttcgctggaaaacatcctgaaaaacctggaccgcgtgaaaccggaatccgttcgtgaacgcattaaagcacacctggaagatctgaaactgagcttggaactgagccgtgtacgctctgacctgccgctcgaagtagatttcgctcgtcgtcgtgaacctgatcgtgaaggcctgcgcgcgttcctggaacgcctggaattcggtagcctgctgcacgagtttggcctgctggaggcgccagcgccgctggaagaagcaccgtggccgccaccggaaggtgcgttcgtaggttttgtcctgtcccgtccggaaccgatgtgggccgaacttaaggctctggcagcttgcaaagaaggccgtgtccaccgcgctaaagatccactggcgggccttaaagacctgaaagaagttcgcggccttctggctaaagacctggcggttctggcgctgcgtgaaggtctggatctcgcgcctagcgatgacccgatgctgttggcatacctgctggacccgagtaacaccaccccggaaggcgttgcccgtcgctatggcggcgaatggactgaggatgctgcccaccgcgcgctgctcgcagaacgcctgcagcagaacttgctggagcgcttgaaaggtgaagaaaaactgctttggctgtatcaggaagttgaaaaaccgctgagccgcgttctggcacacatggaagcgactggcgttcgtctggacgtggcgtacctgaaagctctgtccctggaactggctgaagaaatccgccgcttggaggaagaagttttccgcctggcgggccacccgttcaacctgaacagccgcgaccaactggaacgtgttctgtttgatgaactgcgcctgccggctctgggcaaaacccaaaaaaccggtaagcgttctacttccgccgcggttctggaagcactgcgcgaagcacacccgattgtggaaaaaattctgcagcaccgtgaactgactaaactgaaaaactactatgttgacccgctgccgggcctggttcacccacgtactggtcgcctgcatacccgtttcaaccagaccgcaactgccaccggccgtttgtcgtccagcgatccgaacctgcagaatatcccgatccgcaccccgctgggtcagcgtatccgtcgcgccttcgtcgcagaagccggttgggcgcttgttgcgctggactatagccagattgaactgcgtgttctggctcatctgagcggtgacgagaacctgattcgtgtgtttcaagaaggcaaagatatccacacccagaccgctagctggatgtttggtgtgagcccggaagcggttgatccactgatgcgccgcgcggcgaaaacggtgaactttggtgttctgtacggtatgagcgcgcaccgtttgtctcaggaactggctatcccgtacgaagaagcagttgcgttcattgaacgttatttccagtccttcccgaaagttcgtgcgtggatcgaaaaaaccctggaagaaggtcgtaaacgtggttacgttgaaaccctgttcggccgccgtcgctatgtgccggacctgaacgcgcgtgttaaatctgtacgcgaagcagctgaacgtatggcatttaacatgccggtgcagggtaccgcggctgacctgatgaaactggcgatggttaaactgttcccgcacctgcgcgaaatgggcgcgcgtatgctgctgcaggtgcacgacgaactgctgctggaagcaccgcaggcgcgtgcagaagaagtggcggcgctggcaaaagaagccatggaaaaagcatacccgctggctgtgccgctggaagttgaagtgggtatcggtgaagattggctgagcgctaaaggt

SEQ ID NO: 3 - protein, 554 aa, Synthetic construct
MGLLHEFGLLEAPAPLEEAPWPPPEGAFVGFVLSRPEPMWAELKALAACKEGRVHRAKDPLAGLKDLKEVRGLLAKDLAVLALREGLDLAPSDDPMLLAYLLDPSNTTPEGVARRYGGEWTEDAAHRALLAERLQQNLLERLKGEEKLLWLYQEVEKPLSRVLAHMEATGVRLDVAYLKALSLELAEEIRRLEEEVFRLAGHPFNLNSRDQLERVLFDELRLPALGKTQKTGKRSTSAAVLEALREAHPIVEKILQHRELTKLKNYYVDPLPGLVHPRTGRLHTRFNQTATATGRLSSSDPNLQNIPIRTPLGQRIRRAFVAEAGWALVALDYSQIELRVLAHLSGDKNLIRVFQEGKDIHTQTASWMFGVSPEAVDPLMRRAAKTVNFGVLYGMSAHRLSQELAIPYEEAVAFIERYFQSFPKVRAWLKKTLEEGRKRGYVETLFGRRRYVPDLNARVKSVRRRAERMAFNMPVQGTAADLMKLAMVKLFPHLREMGARMLLQVHDELLLEAPQARAEEVAALAKEAMEKAYPLAVPLEVEVGIGEDWLSAKG

SEQ ID NO: 4 - DNA, 1662 bp, synthetic construct
  source 1..1662: mol_type = other DNA
atgggtctgctgcacgagtttggcctgctggaggcgccagcgccgctggaagaagcaccgtggccgccaccggaaggtgcgttcgtaggttttgtcctgtcccgtccggaaccgatgtgggccgaacttaaggctctggcagcttgcaaagaaggccgtgtccaccgcgctaaagatccactggcgggccttaaagacctgaaagaagttcgcggccttctggctaaagacctggcggttctggcgctgcgtgaaggtctggatctcgcgcctagcgatgacccgatgctgttggcatacctgctggacccgagtaacaccaccccggaaggcgttgcccgtcgctatggcggcgaatggactgaggatgctgcccaccgcgcgctgctcgcagaacgcctgcagcagaacttgctggagcgcttgaaaggtgaagaaaaactgctttggctgtatcaggaagttgaaaaaccgctgagccgcgttctggcacacatggaagcgactggcgttcgtctggacgtggcgtacctgaaagctctgtccctggaactggctgaagaaatccgccgcttggaggaagaagttttccgcctggcgggccacccgttcaacctgaacagccgcgaccaactggaacgtgttctgtttgatgaactgcgcctgccggctctgggcaaaacccaaaaaaccggtaagcgttctacttccgccgcggttctggaagcactgcgcgaagcacacccgattgtggaaaaaattctgcagcaccgtgaactgactaaactgaaaaactactatgttgacccgctgccgggcctggttcacccacgtactggtcgcctgcatacccgtttcaaccagaccgcaactgccaccggccgtttgtcgtccagcgatccgaacctgcagaatatcccgatccgcaccccgctgggtcagcgtatccgtcgcgccttcgtcgcagaagccggttgggcgcttgttgcgctggactatagccagattgaactgcgtgttctggctcatctgagcggtgacaagaacctgattcgtgtgtttcaagaaggcaaagatatccacacccagaccgctagctggatgtttggtgtgagcccggaagcggttgatccactgatgcgccgcgcggcgaaaacggtgaactttggtgttctgtacggtatgagcgcgcaccgtttgtctcaggaactggctatcccgtacgaagaagcagttgcgttcattgaacgttatttccagtccttcccgaaagttcgtgcgtggctcaaaaaaaccctggaagaaggtcgtaaacgtggttacgttgaaaccctgttcggccgccgtcgctatgtgccggacctgaacgcgcgtgttaaatctgtacgccgccgtgctgaacgtatggcatttaacatgccggtgcagggtaccgcggctgacctgatgaaactggcgatggttaaactgttcccgcacctgcgcgaaatgggcgcgcgtatgctgctgcaggtgcacgacgaactgctgctggaagcaccgcaggcgcgtgcagaagaagtggcggcgctggcaaaagaagccatggaaaaagcatacccgctggctgtgccgctggaagttgaagtgggtatcggtgaagattggctgagcgctaaaggt

SEQ ID NO: 5 - protein, 563 aa, synthetic construct
MGLLHEFGLLEAPAPLEEAPWPPPEGAFVGFVLSRPEPMWAELKALAACKEGRVHRAKDPLAGLKDLKEVRGLLAKDLAVLALREGLDLAPSDDPMLLAYLLDPSNTTPEGVARRYGGEWTEDAAHRALLAERLQQNLLERLKGEEKLLWLYQEVEKPLSRVLAHMEATGVRLDVAYLKALSLELAEEIRRLEEEVFRLAGHPFNLNSRDQLERVLFDELRLPALGKTQKTGKRSTSAAVLEALREAHPIVEKILQHRELTKLKNYYVDPLPGLVHPRTGRLHTRFNQTATATGRLSSSDPNLQNIPIRTPLGQRIRRAFVAEAGWALVALDYSQIELRVLAHLSGDKNLIRVFQEGKDIHTQTASWMFGVSPEAVDPLMRRAAKTVNFGVLYGMSAHRLSQELAIPYEEAVAFIERYFQSFPKVRAWLKKTLEEGRKRGYVETLFGRRRYVPDLNARVKSVRRRAERMAFNMPVQGTAADLMKLAMVKLFPHLREMGARMLLQVHDELLLEAPQARAEEVAALAKEAMEKAYPLAVPLEVEVGIGEDWLSAKGLEHHHHHHH

SEQ ID NO: 6 - DNA, 1905 bp, synthetic construct
  source 1..1905: mol_type = other DNA
atgggtctgctgcacgagtttggcctgctggaggcgccagcgccgctggaagaagcaccgtggccgccaccggaaggtgcgttcgtaggttttgtcctgtcccgtccggaaccgatgtgggccgaacttaaggctctggcagcttgcaaagaaggccgtgtccaccgcgctaaagatccactggcgggccttaaagacctgaaagaagttcgcggccttctggctaaagacctggcggttctggcgctgcgtgaaggtctggatctcgcgcctagcgatgacccgatgctgttggcatacctgctggacccgagtaacaccaccccggaaggcgttgcccgtcgctatggcggcgaatggactgaggatgctgcccaccgcgcgctgctcgcagaacgcctgcagcagaacttgctggagcgcttgaaaggtgaagaaaaactgctttggctgtatcaggaagttgaaaaaccgctgagccgcgttctggcacacatggaagcgactggcgttcgtctggacgtggcgtacctgaaagctctgtccctggaactggctgaagaaatccgccgcttggaggaagaagttttccgcctggcgggccacccgttcaacctgaacagccgcgaccaactggaacgtgttctgtttgatgaactgcgcctgccggctctgggcaaaacccaaaaaaccggtaagcgttctacttccgccgcggttctggaagcactgcgcgaagcacacccgattgtggaaaaaattctgcagcaccgtgaactgactaaactgaaaaactactatgttgacccgctgccgggcctggttcacccacgtactggtcgcctgcatacccgtttcaaccagaccgcaactgccaccggccgtttgtcgtccagcgatccgaacctgcagaatatcccgatccgcaccccgctgggtcagcgtatccgtcgcgccttcgtcgcagaagccggttgggcgcttgttgcgctggactatagccagattgaactgcgtgttctggctcatctgagcggtgacaagaacctgattcgtgtgtttcaagaaggcaaagatatccacacccagaccgctagctggatgtttggtgtgagcccggaagcggttgatccactgatgcgccgcgcggcgaaaacggtgaactttggtgttctgtacggtatgagcgcgcaccgtttgtctcaggaactggctatcccgtacgaagaagcagttgcgttcattgaacgttatttccagtccttcccgaaagttcgtgcgtggctcaaaaaaaccctggaagaaggtcgtaaacgtggttacgttgaaaccctgttcggccgccgtcgctatgtgccggacctgaacgcgcgtgttaaatctgtacgccgccgtgctgaacgtatggcatttaacatgccggtgcagggtaccgcggctgacctgatgaaactggcgatggttaaactgttcccgcacctgcgcgaaatgggcgcgcgtatgctgctgcaggtgcacgacgaactgctgctggaagcaccgcaggcgcgtgcagaagaagtggcggcgctggcaaaagaagccatggaaaaagcatacccgctggctgtgccgctggaagttgaagtgggtatcggtgaagattggctgagcgctaaaggtgtcgacgggggcggtggtagcgcaaccgttaaattcaaatacaaaggtgaagaaaaagaagttgatattagcaaaatcaagaaagtttggcgtgttggaaaaatgattagctttacctatgatgaaggtggtgggaaaaccggtcgtggtgcagttagcgaaaaagatgcaccgaaagaactgttacaaatgctggaaaaacagaagaaactcgagcaccaccaccaccaccaccaccactaa

SEQ ID NO: 7 - protein, 634 aa, synthetic construct
MGLLHEFGLLEAPAPLEEAPWPPPEGAFVGFVLSRPEPMWAELKALAACKEGRVHRAKDPLAGLKDLKEVRGLLAKDLAVLALREGLDLAPSDDPMLLAYLLDPSNTTPEGVARRYGGEWTEDAAHRALLAERLQQNLLERLKGEEKLLWLYQEVEKPLSRVLAHMEATGVRLDVAYLKALSLELAEEIRRLEEEVFRLAGHPFNLNSRDQLERVLFDELRLPALGKTQKTGKRSTSAAVLEALREAHPIVEKILQHRELTKLKNYYVDPLPGLVHPRTGRLHTRFNQTATATGRLSSSDPNLQNIPIRTPLGQRIRRAFVAEAGWALVALDYSQIELRVLAHLSGDKNLIRVFQEGKDIHTQTASWMFGVSPEAVDPLMRRAAKTVNFGVLYGMSAHRLSQELAIPYEEAVAFIERYFQSFPKVRAWLKKTLEEGRKRGYVETLFGRRRYVPDLNARVKSVRRRAERMAFNMPVQGTAADLMKLAMVKLFPHLREMGARMLLQVHDELLLEAPQARAEEVAALAKEAMEKAYPLAVPLEVEVGIGEDWLSAKGVDGGGGSATVKFKYKGEEKEVDISKIKKVWRVGKMISFTYDEGGGKTGRGAVSEKDAPKELLQMLEKQKKLEHHHHHHHH

SEQ ID NO: 8 - DNA, 424 bp, unidentified
  source 1..424: mol_type = other DNA
aggttctggtaatgacgaggcgacccgtcgtaccttagctatcgctaagctacgggaggcgaatggtgatcgcggtcagataaatagagaaggtttcttacatgacaaatccttgtcatgggatccggatgttttacaaaccagcatccgtagccttattggcaacctcctctctggctaccgatcgtcgttgtttgggcaatgcacgttctccaacggtgctcctatggggcacaagttgcaggatgcagcgccttacaagaagttcgctgaacaagcaaccgttaccccccgcgctctgagagcggctctattggtccgagaccaatgtgcgccgtggatcagacacgcggtccgctataacgagtcatatgaatttaggctcgttgtagggaacggagtgtttacagttccgaagaataat

SEQ ID NO: 9 - DNA, 40 bp, unidentified
  source 1..40: mol_type = unassigned DNA
ctcatctgagcggtgacaagaacctgattcgtgtgtttca

SEQ ID NO: 10 - DNA, 46 bp, unidentified
  source 1..46: mol_type = unassigned DNA
ctttgtgtgcttagtccaagaacagtggcgagtctactcggtcttg

SEQ ID NO: 11 - DNA, 42 bp, unidentified
  source 1..42: mol_type = unassigned DNA
gaaagttcgtgcgtggctcaaaaaaaccctggaagaaggtcg

SEQ ID NO: 12 - DNA, 47 bp, unidentified
  source 1..47: mol_type = unassigned DNA
gaagaaggtcccaaaaaaactcggtgcgtgcttgaaagcccttcctg

SEQ ID NO: 13 - DNA, 45 bp, unidentified
  source 1..45: mol_type = unassigned DNA
gttaaatctgtacgccgccgtgctgaacgtatggcatttaacatg

SEQ ID NO: 14 - DNA, 47 bp, unidentified
  source 1..47: mol_type = unassigned DNA
tacggtatgcaagtcgtgccgccgcatgtctaaattgtgcgcgcaag

SEQ ID NO: 15 - DNA, 46 bp, unidentified
  source 1..46: mol_type = unassigned DNA
taagaaggagatatacatatgggtctgctgcacgagtttggcctgc

SEQ ID NO: 16 - DNA, 47 bp, unidentified
  source 1..47: mol_type = unassigned DNA
gctaccaccgcccccgtcgacacctttagcgctcagccaatcttcac

SEQ ID NO: 17 - DNA, 40 bp, unidentified
  source 1..40: mol_type = unassigned DNA
gcacgttctccaacggtgctggttgcttgttcagcgaact

SEQ ID NO: 18 - DNA, 41 bp, unidentified
  source 1..41: mol_type = unassigned DNA
gcccaaacaacgacgatcggtaaaaccagcatccgtagcct

SEQ ID NO: 19 - DNA, 19 bp, unidentified
  source 1..19: mol_type = unassigned DNA
tgcaggatgcagcgcctta

SEQ ID NO: 20 - DNA, 19 bp, unidentified
  source 1..19: mol_type = unassigned DNA
ccagagaggaggttgccaa

SEQ ID NO: 21 - DNA, 20 bp, unidentified
  source 1..20: mol_type = unassigned DNA
caatagagccgctctcagag

SEQ ID NO: 22 - DNA, 20 bp, unidentified
  source 1..20: mol_type = unassigned DNA
tgtcatgggatccggatgtt